(19) United States
(12) Patent Application Publication
(10) Pub. No.: US 2025/0120809 A1
(43) Pub. Date: Apr. 17, 2025

(54) REPLACEMENT HEART VALVE DELIVERY SYSTEM

(71) Applicant: BOSTON SCIENTIFIC SCIMED, INC., Maple Grove, MN (US)

(72) Inventors: James Holmberg, Champlin, MN (US); Donal Kyne, Galway (IE); Chris Cullen, Galway (IE); Tim O'Connor, Galway (IE); John Lardner, Galway (IE); James M. Anderson, Corcoran, MN (US); Richard Kiely, Galway (IE); Sean O'Sullivan, Galway (IE)

(73) Assignee: BOSTON SCIENTIFIC SCIMED, INC., MAPLE GROVE, MN (US)

(21) Appl. No.: 18/911,760

(22) Filed: Oct. 10, 2024

Related U.S. Application Data
(60) Provisional application No. 63/543,628, filed on Oct. 11, 2023.

(51) Int. Cl. A61F 2/24

(52) U.S. Cl. CPC A61F 2/2436; A61F 2/2412

(57) ABSTRACT

A replacement heart valve system may include a replacement heart valve implant comprising an expandable framework configured to shift from a radially collapsed configuration to a radially expanded configuration, and a plurality of valve leaflets disposed within and secured to the framework, and an implant delivery system comprising a handle assembly and an elongate shaft assembly extending distally from the handle assembly. The elongate shaft assembly may include an inner shaft axially secured to the handle assembly, an outer tubular member axially secured to the handle assembly and disposed about the inner shaft, and an implant holding portion. The implant holding portion is configured to constrain the implant in the radially collapsed configuration when the implant holding portion is disposed in a delivery configuration, wherein the implant holding portion is rotatable relative to the outer tubular member.

### CROSS-REFERENCE TO RELATED APPLICATIONS

This application claims the benefit of priority of U.S. Provisional Application No. 63/543,628 filed Oct. 11, 2023, the entire disclosure of which is hereby incorporated by reference.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including, artificial heart valves for repair or replacement of diseased heart valves. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, an implant delivery system for delivering a replacement heart valve implant to a native heart valve may comprise a handle assembly, and an elongate shaft assembly extending distally from the handle assembly. The elongate shaft assembly may comprise an inner shaft axially secured to the handle assembly, an outer tubular member axially secured to the handle assembly and disposed about the inner shaft, and an implant holding portion configured to constrain the replacement heart valve implant in a radially collapsed configuration when the implant holding portion is disposed in a delivery configuration, wherein the implant holding portion is rotatable relative to the outer tubular member.

In addition or alternatively to any example described herein, the outer tubular member is rotatable relative to the handle assembly.

In addition or alternatively to any example described herein, the implant delivery system may comprise at least one axial locking assembly configured to rotationally decouple the inner shaft and the outer tubular member from the handle assembly.

In addition or alternatively to any example described herein, the implant delivery system may comprise an intermediate tubular member fixedly secured to the handle assembly.

In addition or alternatively to any example described herein, the inner shaft is movably disposed within the intermediate tubular member and the outer tubular member is movably disposed about the intermediate tubular member.

In addition or alternatively to any example described herein, the outer tubular member is rotatable relative to the inner shaft.

In addition or alternatively to any example described herein, the handle assembly is configured to axially move the inner shaft relative to the outer tubular member.

In addition or alternatively to any example described herein, the implant holding portion comprises a proximal sheath axially secured to the outer tubular member and a distal sheath axially secured to the inner shaft.

In addition or alternatively to any example described herein, the proximal sheath is axially movable relative to the distal sheath to shift the implant holding portion from the delivery configuration to a release configuration.

In addition or alternatively to any example described herein, the implant delivery system may comprise a positioning tube disposed about the outer tubular member.

In addition or alternatively to any example described herein, the positioning tube is axially secured to the handle assembly.

In addition or alternatively to any example described herein, a replacement heart valve system may comprise a replacement heart valve implant comprising an expandable framework configured to shift from a radially collapsed configuration to a radially expanded configuration, and a plurality of valve leaflets disposed within and secured to the expandable framework; and an implant delivery system comprising a handle assembly and an elongate shaft assembly extending distally from the handle assembly. The elongate shaft assembly may comprise an inner shaft axially secured to the handle assembly, an outer tubular member axially secured to the handle assembly and disposed about the inner shaft, and an implant holding portion. The implant holding portion may be configured to constrain the replacement heart valve implant in the radially collapsed configuration when the implant holding portion is disposed in a delivery configuration. The implant holding portion may be rotatable relative to the outer tubular member.

In addition or alternatively to any example described herein, the replacement heart valve implant is configured to shift toward the radially expanded configuration when the implant holding portion moves from the delivery configuration to a release configuration.

In addition or alternatively to any example described herein, the elongate shaft assembly comprises a distal tip fixedly secured to a distal end of the inner shaft.

In addition or alternatively to any example described herein, the implant holding portion comprises a proximal sheath axially secured to the outer tubular member and a distal sheath axially secured to the distal tip.

In addition or alternatively to any example described herein, in the release configuration a distal end of the proximal sheath is spaced apart from a proximal end of the distal sheath by a greater distance than in the delivery configuration.

In addition or alternatively to any example described herein, the elongate shaft assembly further comprises a positioning tube disposed about the outer tubular member.

In addition or alternatively to any example described herein, the positioning tube is axially secured to the handle assembly.

In addition or alternatively to any example described herein, a method of delivering a replacement heart valve implant to a native heart valve may comprise: advancing an implant delivery system in a delivery configuration to a position adjacent the native heart valve, wherein the implant delivery system comprises a handle assembly and an elongate shaft assembly extending distally from the handle assembly, the elongate shaft assembly comprising an inner shaft axially secured to the handle assembly, an intermediate tubular member fixedly attached to the handle assembly, and an outer tubular member axially secured to the handle assembly; wherein the replacement heart valve implant is constrained within an implant holding portion of the implant delivery system in a radially collapsed configuration when the implant delivery system is disposed in the delivery configuration; rotating the handle assembly of the implant delivery system to rotate the replacement heart valve implant within the native heart valve without rotating the outer tubular member; and deploying the replacement heart valve implant within the native heart valve.

In addition or alternatively to any example described herein, rotating the handle assembly of the implant delivery system causes the intermediate tubular member to rotate relative to the outer tubular member.

In addition or alternatively to any example described herein, the implant holding portion comprises a proximal sheath and a distal sheath configured to move axially away from each other to deploy the replacement heart valve implant.

In addition or alternatively to any example described herein, rotating the handle assembly of the implant delivery system causes the intermediate tubular member to rotate relative to the inner shaft.

In addition or alternatively to any example described herein, the distal sheath is axially secured to the inner shaft and the distal sheath is configured to rotate relative to the inner shaft.

In addition or alternatively to any example described herein, the proximal sheath is axially secured to the outer tubular member and the proximal sheath is configured to rotate relative to the outer tubular member.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIGS. 2-3 illustrate selected aspects of a replacement heart valve system;

FIGS. 4-6 are partial cross-sectional views illustrating selected aspects of an implant delivery system of the replacement heart valve system;

FIGS. 7-9 schematically illustrate selected aspects of a method of delivering a replacement heart valve implant to a native heart valve;

FIG. 10 illustrates selected aspects of an axial locking assembly of the implant delivery system of FIGS. 4-6; and

FIG. 11 illustrates selected aspects of an alternative configuration of the axial locking assembly of FIG. 10.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

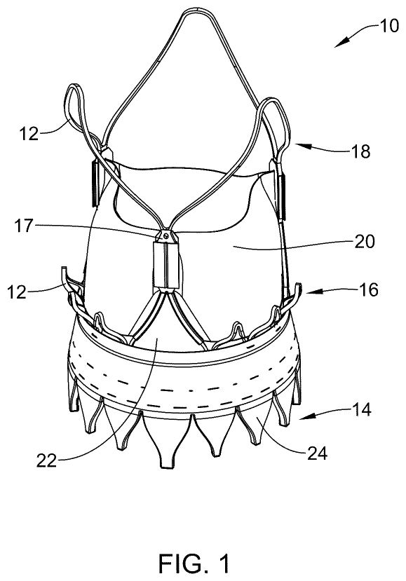

FIG. 1 illustrates selected aspects of a replacement heart valve implant 10. It should be appreciated that the replacement heart valve implant 10 can be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

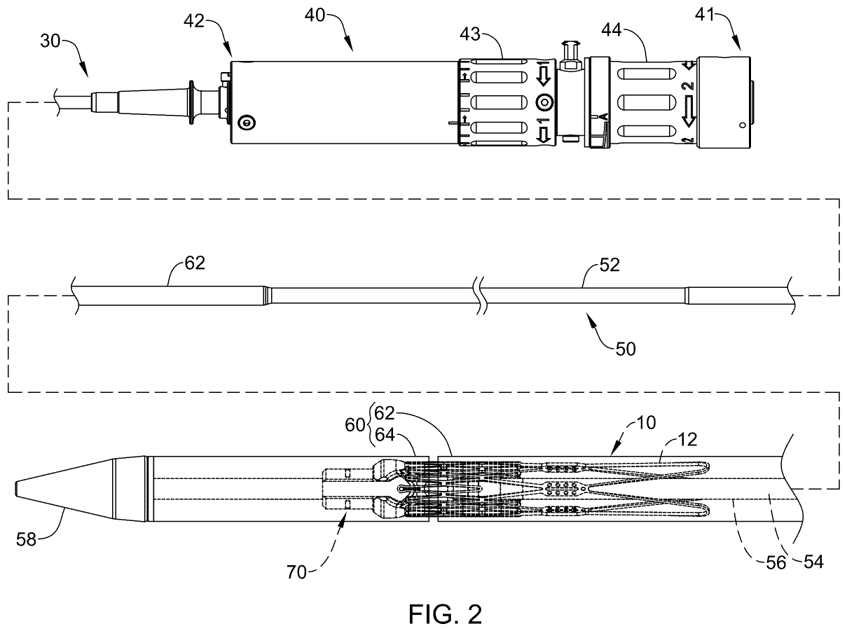

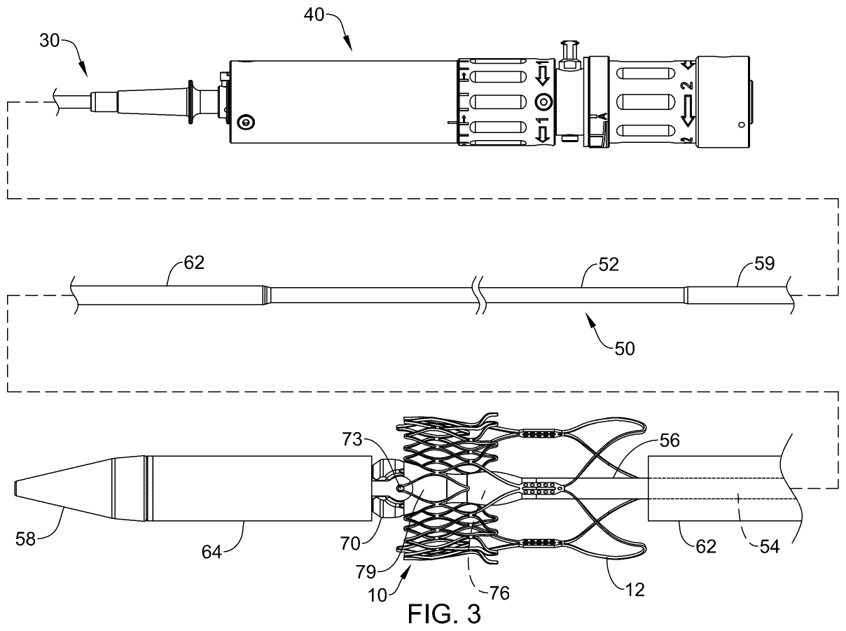

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 and/or the replacement heart valve implant 10 may be configured to shift between a radially collapsed configuration (e.g., FIG. 2) and a radially expanded configuration (e.g., FIGS. 1 and 3). In some embodiments, the expandable framework 12 may be self-expanding from the radially collapsed configuration to the radially expanded configuration. In some embodiments, the expandable framework 12 may be self-biased toward the radially expanded configuration. In some alternative embodiments, the expandable framework 12 may be mechanically expandable from the radially collapsed configuration to the radially expanded configuration. In some alternative embodiments, the expandable framework 12 may be balloon expandable from the radially collapsed configuration to the radially expanded configuration. Other configurations are also contemplated. In some embodiments, the expandable framework 12 may include and/or define a plurality of interstices (e.g., openings) through the expandable framework 12.

In some embodiments, the expandable framework 12 may include and/or define a lower crown 14 proximate an inflow end, an upper crown 16 proximate an outflow end, and a plurality of stabilization arches 18 extending downstream from the outflow end. In some embodiments, the lower crown 14 may be disposed at the inflow end. In some embodiments, the upper crown 16 may be disposed at the outflow end. In some embodiments, the expandable framework 12 may include a tubular wall defining the central lumen, the inflow end, the outflow end, the lower crown 14, and/or the upper crown 16.

In some embodiments, the expandable framework 12 may include and/or define a plurality of commissure posts 17 proximate the outflow end. In some embodiments, the plurality of commissure posts 17 may at least partially define the outflow end. Other configurations are also contemplated. In some embodiments, the plurality of commissure posts 17 may be disposed longitudinally and/or axially between the upper crown 16 and the plurality of stabilization arches 18. In some embodiments, the plurality of stabilization arches 18 may extend downstream of and/or away from the upper crown 16 and/or the plurality of commissure posts 17 in a direction opposite the lower crown 14. In some embodiments, the upper crown 16 may be disposed longitudinally and/or axially between the lower crown 14 and the plurality of stabilization arches 18. In some embodiments, the upper crown 16 may be disposed longitudinally and/or axially between the lower crown 14 and the plurality of commissure posts 17.

In some embodiments, the replacement heart valve implant 10 may include a proximal portion and a distal portion. In some embodiments, orientation of the replacement heart valve implant 10 may be related to an implant delivery device and/or a direction of implantation relative to a treatment site (e.g., a native heart valve, the aortic valve, etc.). In some embodiments, the proximal portion may include the outflow end and/or the plurality of stabilization arches 18. In some embodiments, the proximal portion may include the plurality of commissure posts 17, the upper crown 16, and/or the plurality of valve leaflets 20. In some embodiments, the distal portion may include the inflow end and/or the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12. In at least some embodiments, the plurality of valve leaflets 20 may be coupled, secured, and/or fixedly attached to the expandable framework 12 at the plurality of commissure posts 17 to form and/or define a plurality of commissures.

Each of the plurality of valve leaflets 20 may include a root edge coupled to the expandable framework 12 and a free edge (e.g., a coaptation edge) movable relative to the root edge to coapt with the free edges of the other valve leaflets along a coaptation region. In some embodiments, the plurality of valve leaflets 20 can be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 in a closed position. For example, in some embodiments, the free edges of the plurality of valve leaflets 20 may move into coaptation with one another in the closed position to substantially restrict fluid from flowing through the replacement heart valve implant 10. The free edges of the plurality of valve leaflets 20 may be moved apart from each other in an open position to permit fluid flow through the replacement heart valve implant 10. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from bovine pericardial or other living tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. In some embodiments, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some embodiments, the inner skirt 22 may include a polymer, such as a thermoplastic polymer. In some embodiments, the inner skirt 22 may include at least 50 percent by weight of a polymer. In some embodiments, the outer skirt 24 may include a polymer, such as a thermoplastic polymer. In some embodiments, the outer skirt 24 may include at least 50 percent by weight of a polymer. In some embodiments, one or more of the plurality of valve leaflets 20, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., bovine pericardial, etc.). In some embodiments, the inner skirt 22 and/or the outer skirt 24 may be formed from a coated fabric material. In some embodiments, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 and/or the plurality of frame struts using one or more methods including but not limited to tying with sutures or filaments, adhesive bonding, melt bonding, embedding or over molding, welding, etc.

In some embodiments, the replacement heart valve implant 10 may include a sealing member disposed on the expandable framework 12 proximate the inflow end. In some embodiments, the sealing member may include and/or may be the inner skirt 22. In some embodiments, the sealing member may include and/or may be the outer skirt 24. In some embodiments, the sealing member may include and/or may be the inner skirt 22 and the outer skirt 24. Other configurations are also contemplated.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some embodiments, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated.

FIG. 2-6 illustrate selected aspects of a replacement heart valve system including the replacement heart valve implant 10 and an implant delivery system 30 for delivering a replacement heart valve implant to a native heart valve (e.g., the aortic valve). The implant delivery system 30 may be compatible with and/or usable with the replacement heart valve implant 10. In FIG. 2, some elements that would be hidden from view are shown in phantom to show relative positioning. In FIGS. 2-3, only the expandable framework 12 of the replacement heart valve implant 10 is shown. Other elements of the replacement heart valve implant 10 have been omitted to improve clarity. It should also be noted that FIGS. 2-3 include at least one change of scale (e.g., all parts of the figure are not drawn to the same scale) to improve viewability and show additional detail of selected aspects of the implant delivery system 30.

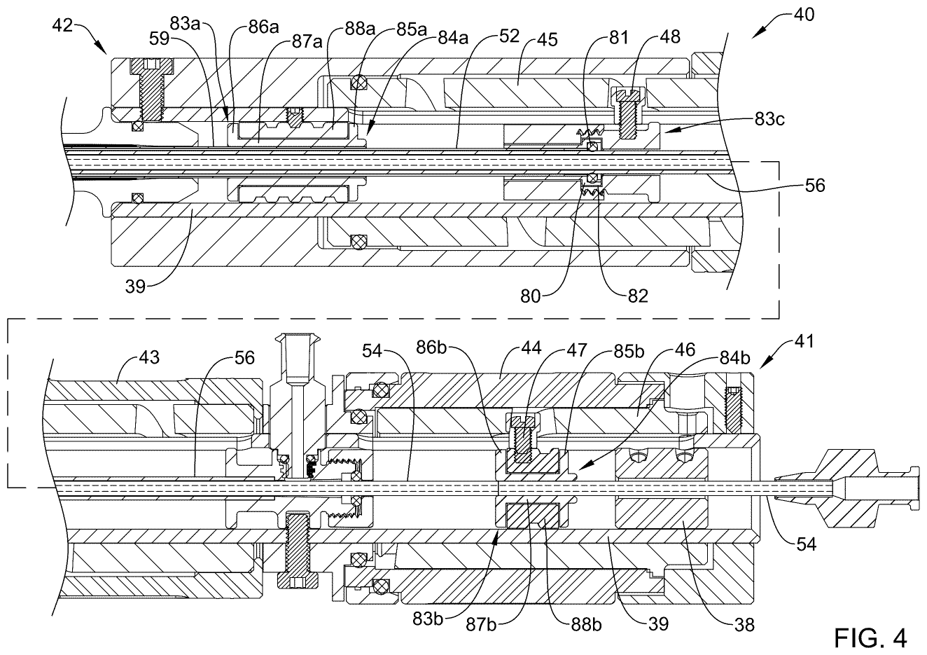

The implant delivery system 30 may include a handle assembly 40 and an elongate shaft assembly 50 extending distally from the handle assembly 40. The handle assembly 40 may include a first end 41 and a second end 42 opposite the first end 41. The elongate shaft assembly 50 may extend distally from the second end 42 of the handle assembly 40. The handle assembly 40 may include a tubular core member 39 (e.g., FIG. 4) disposed therein. The handle assembly 40 may include one or more rotatable knobs. In some embodiments, the one or more rotatable knobs may include a first rotatable knob 43 and a second rotatable knob 44. In at least some embodiments, the first rotatable knob 43 and/or the second rotatable knob 44 may be configured to rotate about a central longitudinal axis of the implant delivery system 30 and/or the handle assembly 40. In some embodiments, the first rotatable knob 43 and/or the second rotatable knob 44 may be configured to rotate about the tubular core member 39.

In some embodiments, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration, as seen in FIG. 2.

In some embodiments, the elongate shaft assembly 50 may comprise an inner shaft 54 axially secured to the handle assembly 40 and/or the tubular core member 39. In some embodiments, the elongate shaft assembly 50 may comprise an outer tubular member 52 axially secured to the handle assembly 40 and disposed about the inner shaft 54. In the context of this disclosure, “axially secured” (and/or variants thereof) generally means that the feature or element is not free floating in an axial direction relative to another feature or element. For example, the feature or element may not be permitted to move freely in an axial direction or on its own but in some embodiments may be movable in an axial direction using a means or mechanism for controlled movement. In some embodiments, the inner shaft 54 may be slidably disposed within a lumen of the outer tubular member 52.

In some embodiments, the inner shaft 54 may be rotationally decoupled from the handle assembly 40 and/or the tubular core member 39. In some embodiments, the outer tubular member 52 may be rotationally decoupled from the handle assembly 40 and/or the tubular core member 39. In the context of this disclosure, “rotationally decoupled” (and/or variants thereof) generally means that the feature or element is permitted to rotate independently of and/or relative to another feature or element. For example, rotation of the handle assembly 40 and/or the tubular core member 39 may not be transferred to the inner shaft 54 and/or the outer tubular member 52, or the inner shaft 54 and/or the outer tubular member 52 does not rotate if/when the handle assembly 40 and/or the tubular core member 39 is rotated.

In some embodiments, the elongate shaft assembly 50 may comprise a distal tip 58 fixedly secured to a distal end of the inner shaft 54 distal of the implant holding portion 60. In some embodiments, the inner shaft 54 may extend distally from the handle assembly 40 within the outer tubular member 52 to the distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the elongate shaft assembly 50 may include an intermediate tubular member 56 disposed within and/or radially inward of the outer tubular member 52 and about and/or radially outward of the inner shaft 54. In some embodiments, the intermediate tubular member 56 may be fixedly attached to the handle assembly 40. In some embodiments, the inner shaft 54 may be slidably disposed within a lumen of the outer tubular member 52 and/or the intermediate tubular member 56. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be axially secured to the outer tubular member 52. In some embodiments, the proximal sheath 62 may be rotationally decoupled from the outer tubular member 52. In some embodiments, the proximal sheath 62 may be axially secured to, rotationally decoupled from, and/or may extend distally from a distal end of the outer tubular member 52. In some embodiments, the distal tip 58 may be fixedly attached to the inner shaft 54. In some embodiments, the distal sheath 64 may be axially secured to the distal tip 58 and/or the inner shaft 54. In some embodiments, the distal sheath 64 may be axially secured to the inner shaft 54 via the distal tip 58 (e.g., the distal sheath 64 is axially secured to the distal tip 58 which is fixedly attached to the inner shaft 54). In some embodiments, the distal sheath 64 may be rotationally decoupled from the distal tip 58 and/or the inner shaft 54. In some alternative embodiments, the distal sheath 64 may be fixedly attached to the inner shaft 54 and/or the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle assembly 40.

In some embodiments, the handle assembly 40 may be configured to manipulate and/or translate the proximal sheath 62 and/or the distal sheath 64 relative to each other using the first rotatable knob 43 and/or the second rotatable knob 44. In some embodiments, the handle assembly 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle assembly 40 may be configured to manipulate and/or translate the outer tubular member 52 and/or the proximal sheath 62 relative to the elongate shaft assembly 50, the inner shaft 54, the intermediate tubular member 56, and/or the distal sheath 64. In some embodiments, the handle assembly 40 may be configured to axially move the inner shaft 54 relative to the outer tubular member 52 and/or the intermediate tubular member 56. In some embodiments, the handle assembly 40 may be configured to axially move the outer tubular member 52 relative to the inner shaft 54 and/or the intermediate tubular member 56.

During delivery of the replacement heart valve implant 10 to a treatment site (e.g., the native heart valve, the aortic valve, etc.), the replacement heart valve implant 10 and/or the expandable framework 12 may be disposed at least partially within the proximal sheath 62 and/or the distal sheath 64 in the radially collapsed configuration when the implant holding portion 60 is disposed in a delivery configuration (e.g., FIG. 2). In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 10 and/or the expandable framework 12 in the radially collapsed configuration when the implant holding portion 60 is disposed in the delivery configuration (e.g., FIG. 2). In some embodiments, the replacement heart valve implant 10 and/or the expandable framework 12 may be releasably coupled to the inner shaft 54, the intermediate tubular member 56, and/or a stent holder 70 (described in more detail below) when the replacement heart valve implant 10 and/or the expandable framework 12 is constrained within the implant holding portion 60 of the implant delivery system 30 in the radially collapsed configuration.

In some embodiments, the proximal sheath 62 may be configured to cover the proximal portion and/or the outflow end of the replacement heart valve implant 10 and/or the expandable framework 12 in the radially collapsed configuration when the implant holding portion 60 is disposed in the delivery configuration, and the distal sheath 64 may be configured to cover the distal portion and/or the inflow end of the replacement heart valve implant 10 and/or the expandable framework 12 in the radially collapsed configuration when the implant holding portion 60 is disposed in the delivery configuration. In some embodiments, the proximal sheath 62 may be disposed adjacent to the distal sheath 64 in the delivery configuration. In some embodiments, the proximal sheath 62 may abut the distal sheath 64 in the delivery configuration. In some embodiments, the proximal sheath 62 may be axially spaced apart from the distal sheath 64 in the delivery configuration. In some embodiments, the proximal sheath 62 may be axially spaced apart from the distal sheath 64 in the delivery configuration by less than 20% of an overall length of the replacement heart valve implant 10 and/or the expandable framework 12. In some embodiments, the proximal sheath 62 may be axially spaced apart from the distal sheath 64 in the delivery configuration by less than 15% of an overall length of the replacement heart valve implant 10 and/or the expandable framework 12. In some embodiments, the proximal sheath 62 may be axially spaced apart from the distal sheath 64 in the delivery configuration by less than 10% of an overall length of the replacement heart valve implant 10 and/or the expandable framework 12. In some embodiments, the proximal sheath 62 may be axially spaced apart from the distal sheath 64 in the delivery configuration by less than 5% of an overall length of the replacement heart valve implant 10 and/or the expandable framework 12. Other configurations are also contemplated.

After advancing the replacement heart valve system and/or the implant delivery system 30 to a position adjacent the native heart valve (e.g., the aortic valve), the replacement heart valve implant 10 and/or the expandable framework 12 may be deployed within the native heart valve (e.g., the aortic valve). In some embodiments, rotation of the replacement heart valve implant 10 may be required to properly align the plurality of commissure posts 17 and/or the plurality of valve leaflets 20 with the corresponding aspects of the native heart valve (e.g., the aortic valve). In order to rotate the replacement heart valve implant 10 within the anatomy, the handle assembly 40 may be rotated about its central longitudinal axis, thereby transmitting torque along the elongate shaft assembly 50 to the replacement heart valve implant 10. Due to the tortuous vasculature that the elongate shaft assembly 50 may be disposed in, friction and/or resistance to rotation may cause torque to build up within the elongate shaft assembly 50. If too much torque builds up, one or more portions of the elongate shaft assembly 50, such as the outer tubular member 52 for example, may fracture or fail. As a result, it may be desirable to transmit torque along the elongate shaft assembly 50 without rotating and/or transmitting the torque to the outer tubular member 52. Accordingly, in some embodiments, the outer tubular member 52 may be rotatable relative to the handle assembly 40, the inner shaft 54, the intermediate tubular member 56, and/or the replacement heart valve implant 10.

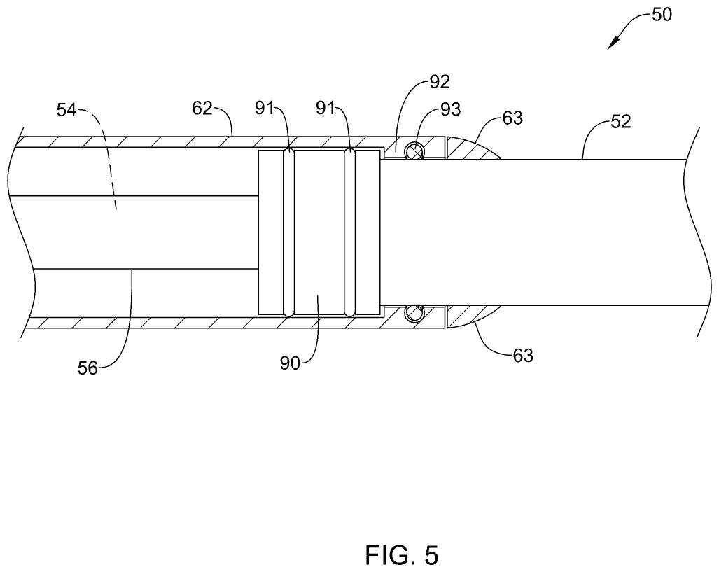

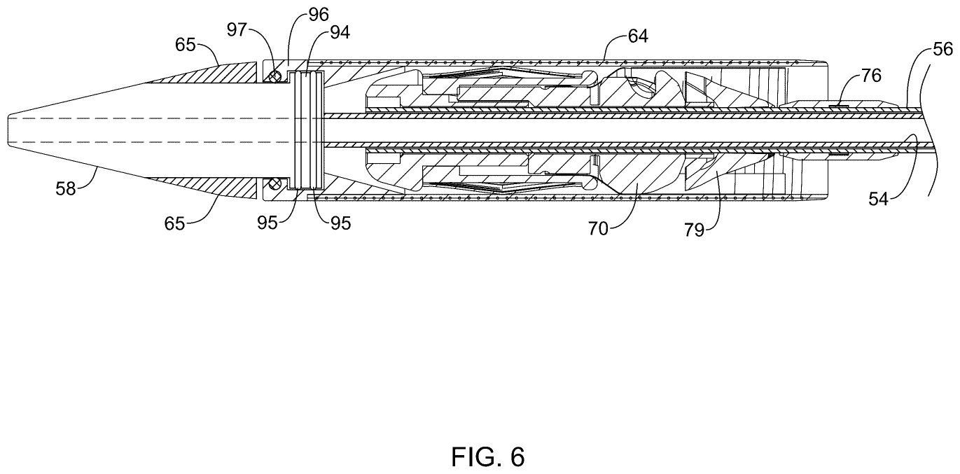

Similarly, friction between the replacement heart valve implant 10 and the implant holding portion 60, the proximal sheath 62, and/or the distal sheath 64 may impede rotation of the replacement heart valve implant 10 and/or may cause damage to the replacement heart valve implant 10 as the replacement heart valve implant 10 is rotated if the implant holding portion 60, the proximal sheath 62, and/or the distal sheath 64 do not also rotate. Accordingly, in some embodiments, the implant holding portion 60 may be rotatable relative to the outer tubular member 52, the intermediate tubular member 56, and/or the inner shaft 54. See FIG. 5 and additional description provided herein. In some embodiments, the proximal sheath 62 may be rotatable relative to the outer tubular member 52 and/or the intermediate tubular member 56. In some embodiments, the distal sheath 64 may be rotatable relative to distal tip 58 and/or the inner shaft 54. See FIG. 6 and additional description provided herein. In some embodiments, the distal sheath 64 may be rotatable relative to the outer tubular member 52 and/or the intermediate tubular member 56.

In some embodiments, rotation of the handle assembly 40 may transmit torque and/or rotation to the replacement heart valve implant 10 via the intermediate tubular member 56, thereby causing the replacement heart valve implant 10 to rotate within the implant holding portion 60 relative to the inner shaft 54 and/or the outer tubular member 52. In some embodiments, rotating the handle assembly 40 of the implant delivery system 30 may cause the replacement heart valve implant 10 to rotate within the implant holding portion 60, thereby causing the implant holding portion 60 to rotate relative to the outer tubular member 52.

As discussed herein, the intermediate tubular member 56 may rotate within and/or relative to the outer tubular member 52 because the outer tubular member 52 may be rotatable relative to and/or rotationally decoupled from the handle assembly 40. In some embodiments, the intermediate tubular member 56 may rotate about and/or relative to the inner shaft 54 because the inner shaft 54 may be rotatable relative to and/or rotationally decoupled from the handle assembly 40. In some embodiments, the outer tubular member 52 may be rotatable independently of the handle assembly 40 and/or the inner shaft 54. In some embodiments, the inner shaft 54 may be rotatable independently of the handle assembly 40 and/or the outer tubular member 52. Additionally, the implant holding portion 60 may be rotatable relative to the outer tubular member 52, the intermediate tubular member 56, and/or the inner shaft 54 such that the implant holding portion 60, the proximal sheath 62, and/or the distal sheath 64 may rotate along with the replacement heart valve implant 10 to prevent damage to the replacement heart valve implant 10. In some embodiments, the implant holding portion 60, the proximal sheath 62, and/or the distal sheath 64 may be rotatable independently of the handle assembly 40, the outer tubular member 52, the intermediate tubular member 56, and/or the inner shaft 54.

Deploying the replacement heart valve implant 10 and/or the expandable framework 12 may include shifting the proximal sheath 62 and the distal sheath 64 of the implant holding portion 60 from the delivery configuration (e.g., FIG. 2) to a release configuration (e.g., FIG. 3) to deploy the replacement heart valve implant 10. In some embodiments, shifting the proximal sheath 62 and the distal sheath 64 of the implant holding portion 60 from the delivery configuration to the release configuration may include shifting the proximal sheath 62 and the distal sheath 64 of the implant holding portion 60 axially apart from each other. In some embodiments, the proximal sheath 62 and the distal sheath 64 may be configured to move axially away from each other to deploy the replacement heart valve implant 10. In some embodiments, in the release configuration, a distal end of the proximal sheath 62 may be axially spaced apart from a proximal end of the distal sheath 64 by a greater distance than in the delivery configuration. In some embodiments, in the release configuration, the distal end of the proximal sheath 62 is axially spaced apart from the proximal end of the distal sheath 64 by at least an overall length of the replacement heart valve implant 10.

As seen in FIG. 4, in some embodiments, the handle assembly 40 may comprise a first translation mechanism 45 operatively coupled with the first rotatable knob 43. In some embodiments, the first translation mechanism 45 may be rotatably and/or fixedly secured to the first rotatable knob 43 (e.g., relative rotation between the first translation mechanism 45 and the first rotatable knob 43 is not permitted). The first translation mechanism 45 may include a first helical slot configured to engage with a first extension 48 (shown as and/or including a threaded fastener) coupled to the outer tubular member 52. In some embodiments, the first extension 48 may be coupled with and/or secured to the outer tubular member 52. As the first rotatable knob 43 is rotated about the central longitudinal axis of the handle assembly 40, the first translation mechanism 45 may also rotate and thereby translate the first extension 48 axially within and/or relative to the handle assembly 40. Axial translation of the first extension 48 may cause and/or result in commensurate axial translation of the outer tubular member 52 relative to the handle assembly 40 and/or the intermediate tubular member 56 and/or thereby shift the proximal sheath 62 between the delivery configuration and the release configuration. In FIG. 4, the components related to the first translation mechanism 45 are shown in an intermediate position within the handle assembly 40 corresponding to the proximal sheath 62 being positioned between the delivery configuration and the release configuration (e.g., partially translated).

In some embodiments, the handle assembly 40 may comprise a second translation mechanism 46 operatively coupled with the second rotatable knob 44. In some embodiments, the second translation mechanism 46 may be rotatably and/or fixedly secured to the second rotatable knob 44 (e.g., relative rotation between the second translation mechanism 46 and the second rotatable knob 44 is not permitted). The second translation mechanism 46 may include a second helical slot configured to engage with a second extension 47 (shown as and/or including a threaded fastener) coupled to the inner shaft 54. In some embodiments, the second extension 47 may be axially secured to the inner shaft 54. In some alternative embodiments, the second extension 47 may be fixedly secured to the inner shaft 54. As the second rotatable knob 44 is rotated about the central longitudinal axis of the handle assembly 40, the second translation mechanism 46 may also rotate and thereby translate the second extension 47 axially within and/or relative to the handle assembly 40. Axial translation of the second extension 47 may cause and/or result in commensurate axial translation of the inner shaft 54 relative to the handle assembly 40 and/or the intermediate tubular member 56 and/or thereby shift the distal sheath 64 between the delivery configuration and the release configuration. In FIG. 4, the components related to the second translation mechanism 46 are shown in an intermediate position within the handle assembly 40 corresponding to the distal sheath 64 being positioned between the delivery configuration and the release configuration (e.g., partially translated).

In some embodiments, the implant delivery system 30 and/or the elongate shaft assembly 50 may comprise a positioning tube 59 (e.g., FIGS. 3-4) disposed over and/or about the outer tubular member 52. In some embodiments, the outer tubular member 52 may extend completely through the positioning tube 59. In some embodiments, the positioning tube 59 may provide additional column strength to the outer tubular member 52 for navigation through tortuous vasculature. In some embodiments, the positioning tube 59 may be axially secured to the handle assembly 40 and/or the tubular core member 39. In some embodiments, the outer tubular member 52 may be axially movable within and/or relative to the positioning tube 59. In some embodiments, the positioning tube 59 may permit axial movement of the outer tubular member 52 (e.g., shifting between the delivery configuration and the release configuration) while protecting the vasculature. Other uses and/or benefits are also contemplated. In some embodiments, the positioning tube 59 may be rotatable relative to the handle assembly 40. In some embodiments, the positioning tube 59 may be rotatable independently of the handle assembly 40. In some embodiments, the outer tubular member 52 may be rotatable within and/or relative to the positioning tube 59. In some embodiments, the outer tubular member 52 may be rotationally decoupled from the handle assembly 40 and/or the tubular core member 39.

In some embodiments, the implant delivery system 30 may comprise at least one axial locking assembly disposed within the handle assembly 40 and configured to axially secure individual tubes or elements of the elongate shaft assembly 50 to the handle assembly 40 and/or the tubular core member 39 and/or configured to rotationally decouple individual tubes or elements of the elongate shaft assembly 50 from the handle assembly 40 and/or the tubular core member 39. In some embodiments, the implant delivery system 30 and/or the at least one axial locking assembly may comprise a first axial locking assembly 83a, a second axial locking assembly 83b, and/or a third axial locking assembly 83c, including but not limited to subsets thereof. In some embodiments, the first axial locking assembly 83a may be configured to axial secure the positioning tube 59 to the handle assembly 40 and/or the tubular core member 39. In some embodiments, the first axial locking assembly 83a may be configured to rotationally decouple the positioning tube 59 from the handle assembly 40 and/or the tubular core member 39. In some embodiments, the second axial locking assembly 83b may be configured to axial secure the inner shaft 54 to the handle assembly 40 and/or the tubular core member 39. In some embodiments, the second axial locking assembly 83b may be configured to rotationally decouple the inner shaft 54 from the handle assembly 40 and/or the tubular core member 39. In some embodiments, the third axial locking assembly 83c may be configured to axial secure the outer tubular member 52 to the handle assembly 40 and/or the tubular core member 39. In some embodiments, the third axial locking assembly 83c may be configured to rotationally decouple the outer tubular member 52 from the handle assembly 40 and/or the tubular core member 39.

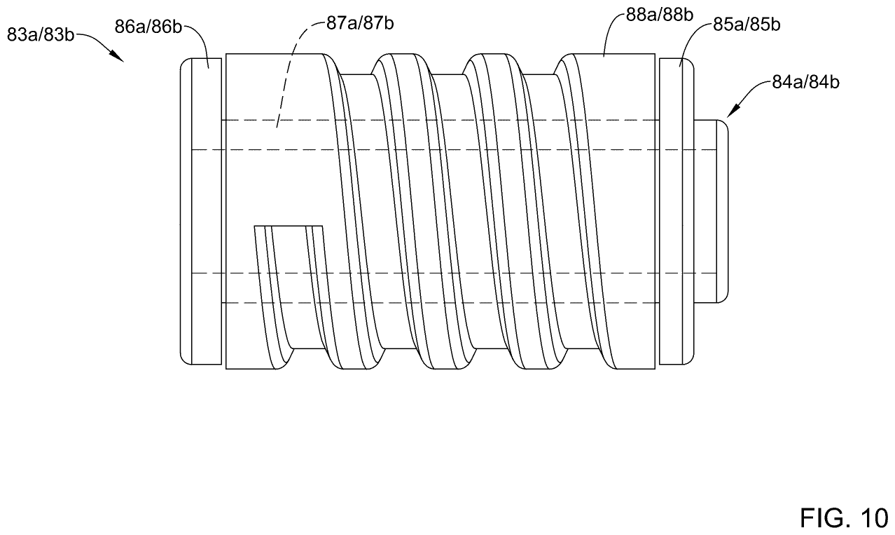

In some embodiments, the first axial locking assembly 83a, shown in greater detail in FIG. 10, may comprise a first axial locking member 84a attached to and extending radially outward from a proximal end of the positioning tube 59. In some embodiments, the first axial locking member 84a may be fixedly attached to the positioning tube 59. In some embodiments, the first axial locking member 84a may comprise a first proximal flange 85a disposed proximate and/or at a proximal end of the first axial locking member 84a. In some embodiments, the first axial locking member 84a may comprise a first distal flange 86a disposed proximate and/or at a distal end of the first axial locking member 84a. The first distal flange 86a may be spaced apart from the first proximal flange 85a. In some embodiments, the first axial locking member 84a may include a first intermediate portion 87a extending between the first proximal flange 85a and the first distal flange 86a. In some embodiments, the first intermediate portion 87a may extend from the first proximal flange 85a to the first distal flange 86a. In at least some embodiments, the first intermediate portion 87a may be annular and/or may be barrel-like. In some embodiments, the positioning tube 59 may be disposed within and/or may be fixedly attached to the first intermediate portion 87a.

In some embodiments, the first axial locking assembly 83a may comprise a first sleeve member 88a configured to be positioned between the first proximal flange 85a and the first distal flange 86a of the first axial locking member 84a such that the first axial locking member 84a is rotatable relative to the first sleeve member 88a. The first sleeve member 88a may be generally annular. In some embodiments, and outer surface of the first sleeve member 88a may comprise a helical groove formed therein. The handle assembly 40 may comprise a set screw 49 configured to engage the tubular core member 39 and the helical groove of the first sleeve member 88a to axially secure the first sleeve member 88a to the handle assembly 40 and/or the tubular core member 39. The helical groove and the set screw 49 may permit adjustment of a relative axial position of the first axial locking assembly 83a within and/or with respect to the tubular core member 39 during initial assembly of the implant delivery system 30.

In at least some embodiments, the first intermediate portion 87a of the first axial locking member 84a may be configured to extend within and/or pass through a lumen of the first sleeve member 88a. In at least some embodiments, an outer diameter of the first proximal flange 85a may be greater than an inner diameter of the first sleeve member 88a, and an outer diameter of the first distal flange 86a may be greater than the inner diameter of the first sleeve member 88a. In some embodiments, the first axial locking assembly 83a may be disposed within the tubular core member 39 and/or the handle assembly 40. In some embodiments, the first sleeve member 88a may be axially trapped between the first proximal flange 85a and the first distal flange 86a such that when the first sleeve member 88a is disposed about and/or around the first intermediate portion 87a of the first axial locking member 84a, and/or when the first intermediate portion 87a of the first axial locking member 84a is disposed within the first sleeve member 88a, the first axial locking member 84a (and the positioning tube 59 attached thereto) is axially secured to the handle assembly 40 and/or the tubular core member 39 (e.g., via the set screw 49, for example). Additionally, when the first sleeve member 88a is disposed about and/or around the first intermediate portion 87a of the first axial locking member 84a, and/or when the first intermediate portion 87a of the first axial locking member 84a is disposed within the first sleeve member 88a, the first axial locking member 84a (and the positioning tube 59 attached thereto) is rotationally decoupled from the handle assembly 40 and/or the tubular core member 39.

In some embodiments, the second axial locking assembly 83b, shown in greater detail in FIG. 10, may comprise a second axial locking member 84b attached to and extending radially outward from a proximal end of the inner shaft 54. In some embodiments, the second axial locking member 84b may be fixedly attached to the inner shaft 54. In some embodiments, the second axial locking member 84b may comprise a second proximal flange 85b disposed proximate and/or at a proximal end of the second axial locking member 84b. In some embodiments, the second axial locking member 84b may comprise a second distal flange 86b disposed proximate and/or at a distal end of the second axial locking member 84b. The second distal flange 86b may be spaced apart from the second proximal flange 85b. In some embodiments, the second axial locking member 84b may include a second intermediate portion 87b extending between the second proximal flange 85b and the second distal flange 86b. In some embodiments, the second intermediate portion 87b may extend from the second proximal flange 85b to the second distal flange 86b. In at least some embodiments, the second intermediate portion 87b may be annular and/or may be barrel-like. In some embodiments, the inner shaft 54 may be disposed within and/or may be fixedly attached to the second intermediate portion 87b.

In some embodiments, the second axial locking assembly 83b may comprise a second sleeve member 88b configured to be positioned between the second proximal flange 85b and the second distal flange 86b of the second axial locking member 84b such that the second axial locking member 84b is rotatable relative to the second sleeve member 88b. The second sleeve member 88b may be generally annular. In at least some embodiments, the second intermediate portion 87b of the second axial locking member 84b may be configured to extend within and/or pass through a lumen of the second sleeve member 88b. In at least some embodiments, an outer diameter of the second proximal flange 85b may be greater than an inner diameter of the second sleeve member 88b, and an outer diameter of the second distal flange 86b may be greater than the inner diameter of the second sleeve member 88b. In some embodiments, the second axial locking assembly 83b may be disposed within the tubular core member 39 and/or the handle assembly 40. As discussed herein, the second extension 47 may be coupled with and/or secured to the inner shaft 54. In some embodiments, the second extension 47 may be coupled with and/or secured to the second axial locking assembly 83b. In some embodiments, the second extension 47 may extend radially outward from the second axial locking assembly 83b to engage with the second helical slot of the second translation mechanism 46. In some embodiments, the second sleeve member 88b may be axially trapped between the second proximal flange 85b and the second distal flange 86b such that when the second sleeve member 88b is disposed about and/or around the second intermediate portion 87b of the second axial locking member 84b, and/or when the second intermediate portion 87b of the second axial locking member 84b is disposed within the second sleeve member 88b, the second axial locking member 84b (and the inner shaft 54 attached thereto) is rotationally decoupled from the handle assembly 40 and/or the tubular core member 39.

In some embodiments, the third axial locking assembly 83c (e.g., FIG. 4) may comprise an annular flange member 80 fixedly attached to and extending radially outward from the outer tubular member 52 proximate a proximal end of the outer tubular member 52. In some embodiments, the annular flange member 80 may be disposed on and/or at the proximal end of the outer tubular member 52. In some embodiments, the third axial locking assembly 83c may include an O-ring 81 disposed within the annular flange member 80 and/or about the intermediate tubular member 56 and/or the outer tubular member 52. In some embodiments, the O-ring 81 may be configured to prevent fluid from passing, such as during a flushing procedure for example. In some embodiments, the third axial locking assembly 83c may comprise an inwardly opening groove 82 configured to receive the annular flange member 80 such that the annular flange member 80 is rotatable within the inwardly opening groove 82. In some embodiments, the annular flange member 80 may extend into the inwardly opening groove 82 such that the annular flange member 80 and/or the outer tubular member 52 is axially secured within and/or relative to the inwardly opening groove 82. In some embodiments, the third axial locking assembly 83c may be disposed within the tubular core member 39 and/or the handle assembly 40. As discussed herein, the first extension 48 may be coupled with and/or secured to the outer tubular member 52. In some embodiments, the first extension 48 may be coupled with and/or secured to the third axial locking assembly 83c. In some embodiments, the first extension 48 may extend radially outward from the third axial locking assembly 83c to engage with the first helical slot of the first translation mechanism 45. In some embodiments, the outer tubular member 52 may be rotationally decoupled from the handle assembly 40 and/or the tubular core member 39.

In some embodiments, the implant holding portion 60 and/or the elongate shaft assembly 50 may include the stent holder 70, seen in FIGS. 2, 3, and 6. In at least some embodiments, the stent holder 70 may be fixedly attached to the elongate shaft assembly 50. In some embodiments, the stent holder 70 may be fixedly attached to the intermediate tubular member 56 of the elongate shaft assembly 50. In some embodiments, the stent holder 70 may be integrally formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the implant delivery system 30. In some embodiments, the stent holder 70 may include at least one projection 73 configured to engage the expandable framework 12 in the radially collapsed configuration. In some embodiments, the at least one projection 73 may be configured to engage the inflow end of the expandable framework 12 in the radially collapsed configuration. In some embodiments, the at least one projection 73 may extend into and/or through interstices of the expandable framework 12. In some embodiments, the expandable framework 12 may include at least one mounting loop configured to receive and/or engage with the at least one projection 73. Other configurations are also contemplated.

The implant delivery system 30 and/or the elongate shaft assembly 50 may include a primary visual indicator 76 (e.g., FIGS. 3 and 6) disposed within the replacement heart valve implant 10 when the replacement heart valve implant 10 and/or the expandable framework 12 is constrained within the implant holding portion 60 in the radially collapsed configuration. The primary visual indicator 76 may be configured and/or adapted to be visible under fluoroscopy with an imaging device. Other imaging means suitable for use with transcatheter surgical procedures are also contemplated. The implant delivery system 30 and/or the primary visual indicator 76 may be configured to cooperate with the imaging device to position the replacement heart valve implant 10 at a desired insertion depth within the native heart valve (e.g., the aortic valve). In some embodiments, the primary visual indicator 76 may be fixedly attached to the elongate shaft assembly 50 and/or the intermediate tubular member 56 by a shrink wrap or by an adhesive element. In some embodiments, the primary visual indicator 76 may be and/or may include a marker band. In some embodiments, the primary visual indicator 76 may be at least partially radiopaque. In some embodiments, the primary visual indicator 76 may be completely radiopaque. Other configurations are also contemplated.

In use, the implant delivery system 30 may be advanced to a position adjacent to the treatment site (e.g., the native heart valve). In one example, the implant delivery system 30 may be advanced through the vasculature and across the aortic arch to a position adjacent to the native heart valve (e.g., the aortic valve). Alternative approaches to treat a defective aortic valve and/or other heart valve(s) are also contemplated with the implant delivery system 30.

The desired insertion depth may be selected to maximize radially outward force of the expandable framework 12 within the native heart valve (e.g., the aortic valve). Positioning the replacement heart valve implant 10 at the desired insertion depth and/or within a maximum tolerance from the desired insertion depth, the replacement heart valve implant 10 and/or the expandable framework 12 may exhibit optimal arching within the native heart valve (e.g., the aortic valve) and thereby prevent migration of the replacement heart valve implant 10 and/or the expandable framework 12 downstream (or upstream).

Positioning the replacement heart valve implant 10 and/or the expandable framework 12 within the native heart valve (e.g., the aortic valve) may be accomplished by locating the primary visual indicator 76 relative to the native heart valve (e.g., the aortic valve). During visualization, the native heart valve (e.g., the aortic valve) may be identified and/or visualized under fluoroscopy using known means and/or methods, such as contrast injection.

In some embodiments, the implant delivery system 30 and/or the elongate shaft assembly 50 may include the stent holder 70 configured to engage the expandable framework 12 of the replacement heart valve implant 10 in the radially collapsed configuration and/or when the replacement heart valve implant 10 is constrained within the implant holding portion 60 of the implant delivery system 30. In some embodiments, the stent holder 70 may include a body, a first end portion extending proximally from the body, and a second end portion disposed opposite the first end portion. In some embodiments, at least a portion of the first end portion may extend radially outward from and/or radially outward of the body. In some embodiments, the first end portion may have a generally bulbous shape. In some embodiments, the stent holder 70 may be configured and/or adapted to be visible under fluoroscopy. In some embodiments, the stent holder 70 may be formed from stainless steel. Some suitable but non-limiting materials for the stent holder 70 and/or components or elements thereof are described below.

In some embodiments, an outermost radial extent of the first end portion of the stent holder 70 may be disposed proximate a distal end of the first end portion of the stent holder 70. In some embodiments, the first end portion of the stent holder 70 may be tapered radially inward in a proximal direction from the outermost radial extent of the stent holder 70. In some embodiments, the stent holder 70 may include a lumen extending longitudinally and/or axially therethrough. In at least some embodiments, at least a portion of the elongate shaft assembly 50 may extend longitudinally and/or axially through the lumen of the stent holder 70.

The first end portion may be configured and/or adapted to engage the expandable framework 12 of the replacement heart valve implant 10 in the radially collapsed configuration and/or when the replacement heart valve implant 10 is constrained within the implant holding portion 60 of the implant delivery system 30. In some embodiments, the first end portion may include the at least one projection 73 configured and/or adapted to engage the expandable framework 12 of the replacement heart valve implant 10 in the radially collapsed configuration and/or when the replacement heart valve implant 10 is constrained within the implant holding portion 60 of the implant delivery system 30. In some embodiments, the at least one projection 73 may extend radially outward from the first end portion of the stent holder 70.

In some embodiments, the implant delivery system 30 and/or the implant holding portion 60 may include an atraumatic transition shield 79 (e.g., FIGS. 3 and 6). The atraumatic transition shield 79 may be disposed adjacent the stent holder 70. In some embodiments, the atraumatic transition shield 79 may be disposed between the stent holder 70 and the handle assembly 40. In some embodiments, the atraumatic transition shield 79 may be disposed proximal of the stent holder 70. In some embodiments, the atraumatic transition shield 79 may be disposed at and/or adjacent the first end portion of the stent holder 70. In some embodiments, the atraumatic transition shield 79 may axially overlap the first end portion of the stent holder 70. In some embodiments, the atraumatic transition shield 79 may be disposed radially outward of at least a portion of the first end portion of the stent holder 70. In some embodiments, the atraumatic transition shield 79 may be tapered radially inward in the downstream direction and/or the proximal direction and/or toward the handle assembly 40. The atraumatic transition shield 79 may be configured to prevent the replacement heart valve implant 10, the expandable framework 12, the plurality of valve leaflets 20, etc. from catching on the stent holder 70 as the implant delivery system 30 is withdrawn after deploying the replacement heart valve implant 10.

In some embodiments, the primary visual indicator 76 may be disposed adjacent a proximal end of the atraumatic transition shield 79. In some embodiments, the primary visual indicator 76 may be disposed downstream and/or proximal of the atraumatic transition shield 79. In some embodiments, the primary visual indicator 76 and the atraumatic transition shield 79 may axially overlap. In some embodiments, the primary visual indicator 76 may be fixedly attached to the elongate shaft assembly 50. In some embodiments, the primary visual indicator 76 may be embedded in the elongate shaft assembly 50 and/or the intermediate tubular member 56. In some embodiments, the primary visual indicator 76 may be secured and/or fixedly attached to the intermediate tubular member 56, for example by adhesive bonding, welding, shrink wrap, etc. Other configurations are also contemplated.

Turning now to FIG. 5, some additional details pertaining to the proximal sheath 62 are illustrated in partial cross-section. In some embodiments, a distal end of the outer tubular member 52 may comprise a proximal annular extension 90 extending radially outward from an outer surface of the outer tubular member 52. In some embodiments, the proximal sheath 62 may be disposed radially outward of the proximal annular extension 90. In some embodiments, the proximal annular extension 90 may be disposed within the proximal sheath 62. In some embodiments, the proximal annular extension 90 may include at least one O-ring 91 (e.g., at least one first O-ring) disposed about the proximal annular extension 90. In some embodiments, the at least one O-ring 91 may be configured to engage the proximal annular extension 90 and the proximal sheath 62. In some embodiments, the at least one O-ring 91 may function as and/or may form a sealing structure between the proximal annular extension 90 and the proximal sheath 62. In some embodiments, the at least one O-ring 91 may function as and/or may form a sliding interface configured to permit the proximal sheath 62 to rotate relative to the outer tubular member 52 and/or the proximal annular extension 90. In some embodiments, the at least one O-ring 91 may be formed from a relatively soft polymeric material such as silicone or a similar material. In some embodiments, the at least one O-ring 91 may be formed from a relatively hard polymeric material, such as PTFE or a similar material, a composite material, etc. Some suitable but non-limiting materials for the at least one O-ring 91 are described below.

In some embodiments, the proximal sheath 62 may comprise a proximal radial extension 92 extending radially inward from an inner surface of the proximal sheath 62 proximate and/or at a proximal end of the proximal sheath 62. In at least some embodiments, the proximal radial extension 92 may extend radially inward of an outer surface of the proximal annular extension 90. In some embodiments, the proximal radial extension 92 may be disposed about and/or around the outer tubular member 52 proximal of the proximal annular extension 90. In some embodiments, the proximal radial extension 92 may include at least one O-ring 93 (e.g., at least one second O-ring) extending radially inward of and/or from the proximal radial extension 92. The at least one O-ring 93 may be disposed about and/or around the outer tubular member 52. In some embodiments, the at least one O-ring 93 may be configured to engage the proximal radial extension 92 and the outer tubular member 52. In some embodiments, the at least one O-ring 93 may function as and/or may form a sealing structure between the proximal radial extension 92 and the outer tubular member 52. In some embodiments, the at least one O-ring 93 may function as and/or may form a sliding interface configured to permit the proximal sheath 62 and/or the proximal radial extension 92 to rotate relative to the outer tubular member 52 and/or the proximal annular extension 90. In some embodiments, the at least one O-ring 93 may be formed from a relatively soft polymeric material such as silicone or a similar material. In some embodiments, the at least one O-ring 93 may be formed from a relatively hard polymeric material, such as PTFE or a similar material, a composite material, etc. Some suitable but non-limiting materials for the at least one O-ring 93 are described below.

In some embodiments, the elongate shaft assembly 50 and/or the outer tubular member 52 may comprise a proximal locking ring 63 extending radially outward from an outer surface of the outer tubular member 52. In some embodiments, the proximal locking ring 63 may have an outer extent greater than an inner diameter of the proximal radial extension 92. The proximal locking ring 63 may be disposed proximal of the proximal annular extension 90 and/or the proximal radial extension 92. In some embodiments, the proximal radial extension 92 may be disposed axially between the proximal locking ring 63 and the proximal annular extension 90. In some embodiments, the proximal locking ring 63 may be fixedly attached to the outer tubular member 52. In some embodiments, the proximal locking ring 63 may be adhesively bonded to an outer surface of the outer tubular member 52. In some embodiments, the proximal locking ring 63 may be welded or melt bonded to the outer surface of the outer tubular member 52. In some alternative configurations, the proximal locking ring 63 may be integrally formed with and/or may be monolithically formed with the outer tubular member 52. In some embodiments, the proximal locking ring 63 may cooperate with the proximal annular extension 90 and the proximal radial extension 92 to axially secure the proximal sheath 62 to the outer tubular member 52 while permitting the proximal radial extension 92 and/or the proximal sheath 62 to rotate about and/or relative to the outer tubular member 52. For example, the proximal radial extension 92 may be unable to shift and/or may be prevented from shifting proximally of the proximal locking ring 63 or distally of the proximal annular extension 90, thereby preventing the proximal sheath 62 from translating in an axial direction relative to the outer tubular member 52.

Turning now to FIG. 6, some additional details pertaining to the distal sheath 64 are illustrated in partial cross-section. In some embodiments, a proximal end of the distal tip 58 may comprise a distal annular extension 94 extending radially outward from an outer surface of the distal tip 58. In some embodiments, the distal sheath 64 may be disposed radially outward of the distal annular extension 94. In some embodiments, the distal annular extension 94 may be disposed within the distal sheath 64. In some embodiments, the distal annular extension 94 may include at least one O-ring 95 (e.g., at least one third O-ring) disposed about the distal annular extension 94. In some embodiments, the at least one O-ring 95 may be configured to engage the distal annular extension 94 and the distal sheath 64. In some embodiments, the at least one O-ring 95 may function as and/or may form a sealing structure between the distal annular extension 94 and the distal sheath 64. In some embodiments, the at least one O-ring 95 may function as and/or may form a sliding interface configured to permit the distal sheath 64 to rotate relative to the distal tip 58 and/or the distal annular extension 94. In some embodiments, the at least one O-ring 95 may be formed from a relatively soft polymeric material such as silicone or a similar material. In some embodiments, the at least one O-ring 95 may be formed from a relatively hard polymeric material, such as PTFE or a similar material, a composite material, etc. Some suitable but non-limiting materials for the at least one O-ring 95 are described below.

In some embodiments, the distal sheath 64 may comprise a distal radial extension 96 extending radially inward from an inner surface of the distal sheath 64 proximate and/or at a distal end of the distal sheath 64. In at least some embodiments, the distal radial extension 96 may extend radially inward of an outer surface of the distal annular extension 94. In some embodiments, the distal radial extension 96 may be disposed about and/or around the distal tip 58 distal of the distal annular extension 94. In some embodiments, the distal radial extension 96 may include at least one O-ring 97 (e.g., at least one fourth O-ring) extending radially inward of and/or from the distal radial extension 96. The at least one O-ring 97 may be disposed about and/or around the distal tip 58. In some embodiments, the at least one O-ring 97 may be configured to engage the distal radial extension 96 and the distal tip 58. In some embodiments, the at least one O-ring 97 may function as and/or may form a sealing structure between the distal radial extension 96 and the distal tip 58. In some embodiments, the at least one O-ring 97 may function as and/or may form a sliding interface configured to permit the distal sheath 64 and/or the distal radial extension 96 to rotate relative to the distal tip 58 and/or the distal annular extension 94. In some embodiments, the at least one O-ring 97 may be formed from a relatively soft polymeric material such as silicone or a similar material. In some embodiments, the at least one O-ring 97 may be formed from a relatively hard polymeric material, such as PTFE or a similar material, a composite material, etc. Some suitable but non-limiting materials for the at least one O-ring 97 are described below.

In some embodiments, the elongate shaft assembly 50 and/or the distal tip 58 may comprise a distal locking ring 65 extending radially outward from an outer surface of the distal tip 58. In some embodiments, the distal locking ring 65 may have an outer extent greater than an inner diameter of the distal radial extension 96. The distal locking ring 65 may be disposed distal of the distal annular extension 94 and/or the distal radial extension 96. In some embodiments, the distal radial extension 96 may be disposed axially between the distal locking ring 65 and the distal annular extension 94. In some embodiments, the distal locking ring 65 may be fixedly attached to the distal tip 58. In some embodiments, the distal locking ring 65 may be adhesively bonded to an outer surface of the distal tip 58. In some embodiments, the distal locking ring 65 may be welded or melt bonded to the outer surface of the distal tip 58. In some alternative configurations, the distal locking ring 65 may be integrally formed with and/or may be monolithically formed with the distal tip 58. In some embodiments, the distal locking ring 65 may cooperate with the distal annular extension 94 and the distal radial extension 96 to axially secure the distal sheath 64 to the distal tip 58 while permitting the distal radial extension 96 and/or the distal sheath 64 to rotate about and/or relative to the distal tip 58. For example, the distal radial extension 96 may be unable to shift and/or may be prevented from shifting distally of the distal locking ring 65 or proximally of the distal annular extension 94, thereby preventing the distal sheath 64 from translating in an axial direction relative to the inner shaft 54 and/or the distal tip 58.

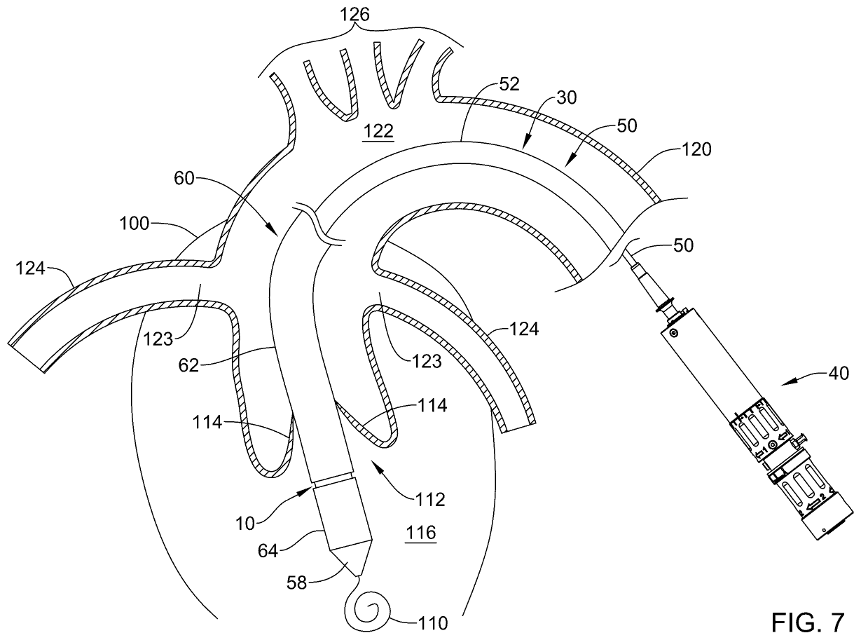

FIGS. 7-9 schematically illustrate selected aspects of a method of delivering the replacement heart valve implant 10 to a native heart valve (e.g., the aortic valve). The method of delivering the replacement heart valve implant 10 to the native heart valve (e.g., the aortic valve) may comprise advancing the implant delivery system 30 to a position adjacent the native heart valve (e.g., the aortic valve), as seen in FIG. 7. In at least some embodiments, the method may comprise advancing the implant delivery system 30 to a position adjacent the native heart valve (e.g., the aortic valve) over a guidewire 110, which may have been previously navigated through the patient's vasculature.

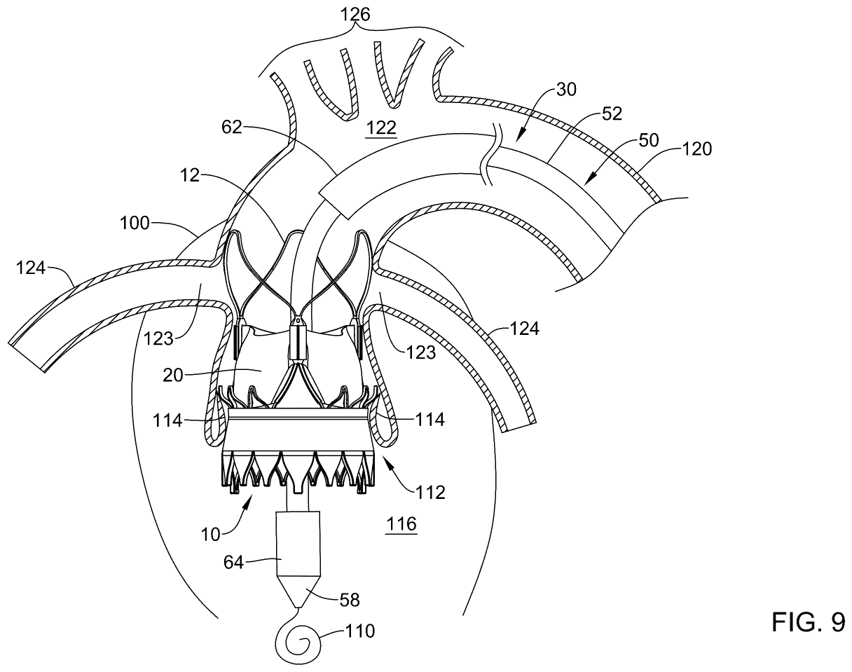

As discussed herein, the replacement heart valve implant 10 may be constrained within the implant holding portion 60 of the implant delivery system 30 in the radially collapsed configuration when the implant delivery system is in the delivery configuration, as seen in FIG. 2. The method of delivering the replacement heart valve implant 10 to the native heart valve (e.g., the aortic valve) may further comprise deploying the replacement heart valve implant 10 and/or the expandable framework 12 within the native heart valve (e.g., the aortic valve), as seen in FIG. 9.

For reference, FIGS. 7-9 illustrates schematic partial cut-away views of a portion of a patient's heart 100 including the aortic valve 112 having native valve leaflets 114 disposed within and/or extending from the native valve annulus, a left ventricle 116, and certain connected vasculature, such as the aorta 120 connected to the aortic valve 112 of the patient's heart 100 by the aortic arch 122, the coronary arteries 124, the ostia 123 of the coronary arteries 124, and other large arteries 126 (e.g., subclavian arteries, carotid arteries, brachiocephalic artery) that extend from the aortic arch 122 to important internal organs. For the purpose of this disclosure, the discussion herein is directed toward use in treating the aortic valve 112 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure. Additionally, the views include at least one change of scale.

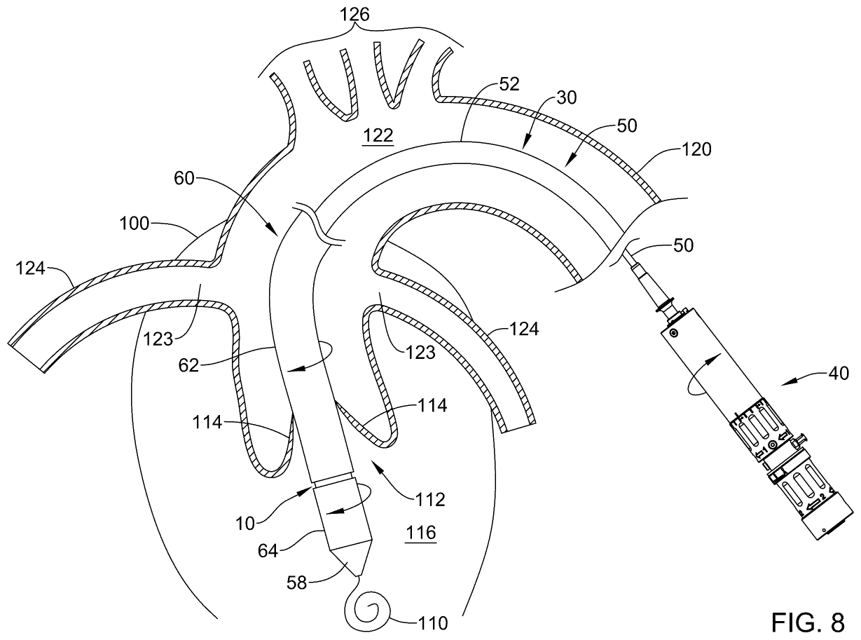

In some embodiments, the method may comprise rotating the handle assembly 40 of the implant delivery system 30 to rotate the replacement heart valve implant 10 within the native heart valve (e.g., the aortic valve 112), as seen in FIG. 8. In some embodiments, the method may comprise rotating the handle assembly 40 of the implant delivery system 30 to rotate the replacement heart valve implant 10 within the native heart valve (e.g., the aortic valve 112) without rotating the outer tubular member 52 and/or the positioning tube 59. In some embodiments, rotating the handle assembly 40 may cause the intermediate tubular member 56 to rotate relative to the outer tubular member 52. In some embodiments, rotating the handle assembly 40 may cause the intermediate tubular member 56 to rotate relative to the inner shaft 54. In some embodiments, rotating the handle assembly 40 may cause the intermediate tubular member 56 to rotate relative to the outer tubular member 52 and the inner shaft 54. In some embodiments, rotating the handle assembly 40 may cause the inner shaft 54 to rotate relative to the outer tubular member 52 and/or the intermediate tubular member 56.

In some embodiments, rotating the handle assembly 40 of the implant delivery system 30 may cause the implant holding portion 60 to rotate relative to the outer tubular member 52. In some embodiments, rotating the handle assembly 40 of the implant delivery system 30 may cause the implant holding portion 60 to rotate relative to the inner shaft 54. In some embodiments, rotating the handle assembly 40 of the implant delivery system 30 may cause the implant holding portion 60 to rotate relative to the outer tubular member 52 and the inner shaft 54.

In some embodiments, deploying the replacement heart valve implant 10 and/or the expandable framework 12 within the native heart valve (e.g., the aortic valve 112) may comprise shifting the implant delivery system 30 from the delivery configuration toward and/or to the release configuration at the position adjacent and/or within the native heart valve (e.g., the aortic valve 112) to deploy the replacement heart valve implant 10 within the native heart valve (e.g., the aortic valve 112).

In some embodiments, shifting the implant delivery system 30 from the delivery configuration toward and/or to the release configuration and/or deploying the replacement heart valve implant 10 and/or the expandable framework 12 within the native heart valve (e.g., the aortic valve 112) may further comprise shifting the proximal sheath 62 of the implant delivery system 30 and/or the implant holding portion 60 downstream and/or proximally relative to the replacement heart valve implant 10 and/or the expandable framework 12 to release the proximal portion of the replacement heart valve implant 10 and/or the expandable framework 12, thereby permitting the proximal portion of the replacement heart valve implant 10 and/or the expandable framework 12 to shift toward the radially expanded configuration.

In some embodiments, shifting the implant delivery system 30 from the delivery configuration toward and/or to the release configuration and/or deploying the replacement heart valve implant 10 and/or the expandable framework 12 within the native heart valve (e.g., the aortic valve 112) may further comprise thereafter, shifting the distal sheath 64 of the implant delivery system 30 and/or the implant holding portion 60 upstream and/or distally relative to the replacement heart valve implant 10 and/or the expandable framework 12 to release the distal portion of the replacement heart valve implant 10 and/or the expandable framework 12, thereby permitting the distal portion of the replacement heart valve implant 10 and/or the expandable framework 12 to shift toward the radially expanded configuration, as seen in FIG. 9.

In some embodiments, after shifting the implant delivery system 30 from the delivery configuration toward and/or to the release configuration and/or deploying the replacement heart valve implant 10 and/or the expandable framework 12 within the native heart valve (e.g., the aortic valve 112), the method may comprise retraction and/or withdrawal of the implant delivery system 30 relative to the replacement heart valve implant 10 and/or the expandable framework 12. In some embodiments, retraction and/or withdrawal of the implant delivery system 30 may include moving and/or translating the implant delivery system 30 downstream and/or proximally relative to the replacement heart valve implant 10 and/or the expandable framework 12. In some alternative configurations, retraction and/or withdrawal of the implant delivery system 30 may include moving and/or translating the implant delivery system 30 upstream and/or distally relative to the replacement heart valve implant 10 and/or the expandable framework 12. Other configurations are also contemplated.

In some embodiments, after deploying the replacement heart valve implant 10 and/or the expandable framework 12 within the native heart valve (e.g., the aortic valve 112), the method may comprise shifting the implant holding portion 60 from the release configuration to the delivery configuration. For example, shifting the implant holding portion 60 from the release configuration to the delivery configuration may include moving and/or translating the proximal sheath 62 and the distal sheath 64 towards each other. In some embodiments, shifting the implant holding portion 60 from the release configuration to the delivery configuration may include moving and/or translating the proximal sheath 62 upstream and/or distally relative to the tubular member (e.g., the intermediate tubular member 56) and/or moving and/or translating the distal sheath 64 downstream and/or proximally relative to the tubular member (e.g., the intermediate tubular member 56).

In some embodiments, after deploying the replacement heart valve implant 10 and/or the expandable framework 12 within the native heart valve (e.g., the aortic valve 112), the method may comprise further retraction and/or withdrawal of the implant delivery system 30 relative to the replacement heart valve implant 10 and/or the expandable framework 12. In some embodiments, after deploying the replacement heart valve implant 10 and/or the expandable framework 12 within the native heart valve (e.g., the aortic valve), the method may comprise retraction and/or withdrawal of the implant delivery system 30 from the treatment site, from the position adjacent the native heart valve (e.g., the aortic valve), and/or from the patient.

In at least some interventions, the replacement heart valve implant 10 may be deployed within the native heart valve (e.g., the native heart valve is left in place and not excised). Alternatively, the native heart valve may be removed (such as through valvuloplasty, for example) and the replacement heart valve implant 10 may be deployed in its place as a replacement.

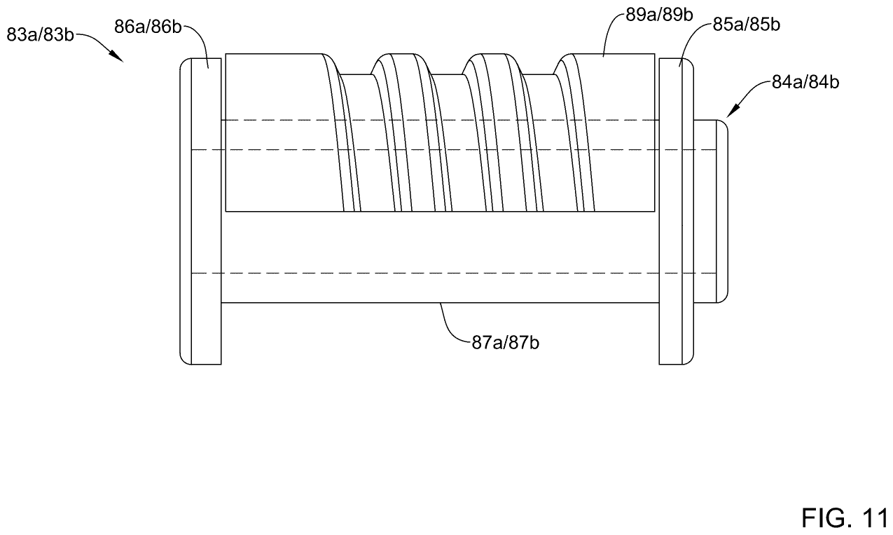

FIG. 10 illustrates selected aspects of the at least one axial locking assembly, the first axial locking assembly 83a, and/or the second axial locking assembly 83b, as discussed herein. FIG. 11 illustrates an alternative configuration for the at least one axial locking assembly, the first axial locking assembly 83a, and/or the second axial locking assembly 83b, wherein the at least one axial locking assembly, the first axial locking assembly 83a, and/or the second axial locking assembly 83b is substantially the same as other embodiments described herein except that the first sleeve member 88a and the second sleeve member 88b have been replaced with a first partial sleeve member 89a and a second partial sleeve member 89b, respectively.

Similar to above, in some embodiments, the first axial locking assembly 83a may comprise a first partial sleeve member 89a configured to be positioned between the first proximal flange 85a and the first distal flange 86a of the first axial locking member 84a such that the first axial locking member 84a is rotatable relative to the first partial sleeve member 89a. In some embodiments, an outer surface of the first partial sleeve member 89a may comprise an angled groove formed therein. In some embodiments, the first partial sleeve member 89a may comprise a threaded aperture formed therein. The handle assembly 40 may comprise a set screw 49 configured to engage the tubular core member 39 and/or the threaded aperture to axially secure the first partial sleeve member 89a to the handle assembly 40 and/or the tubular core member 39. In some embodiments, the angled groove and the set screw 49 may permit adjustment of a relative axial position of the first axial locking assembly 83a within and/or with respect to the tubular core member 39 during initial assembly of the implant delivery system 30.

In some embodiments, the first partial sleeve member 89a may be generally concave in shape and/or may define a partial tube or a partial tubular structure about a longitudinal axis of the first partial sleeve member 89a. The first partial sleeve member 89a extends around and/or along less than 100% of a circumference around the longitudinal axis of the first partial sleeve member 89a. In some embodiments, the first partial sleeve member 89a may extend around and/or along at least 25% of a circumference around the longitudinal axis of the first partial sleeve member 89a. In some embodiments, the first partial sleeve member 89a may extend around and/or along at least 40% of a circumference around the longitudinal axis of the first partial sleeve member 89a. In some embodiments, the first partial sleeve member 89a may extend around and/or along at least 50% of a circumference around the longitudinal axis of the first partial sleeve member 89a. In some embodiments, the first partial sleeve member 89a may extend around and/or along at least 60% of a circumference around the longitudinal axis of the first partial sleeve member 89a. In some embodiments, the first partial sleeve member 89a may extend around and/or along at least 75% of a circumference around the longitudinal axis of the first partial sleeve member 89a.

In at least some embodiments, the first intermediate portion 87a of the first axial locking member 84a may be configured to extend within and/or pass through a concave portion of the first partial sleeve member 89a. In at least some embodiments, an outer diameter of the first proximal flange 85a may be greater than an inner diameter of the concave portion of the first partial sleeve member 89a, and an outer diameter of the first distal flange 86a may be greater than the inner diameter of the concave portion of the first partial sleeve member 89a. In some embodiments, the first axial locking assembly 83a may be disposed within the tubular core member 39 and/or the handle assembly 40. In some embodiments, the first partial sleeve member 89a may be axially trapped between the first proximal flange 85a and the first distal flange 86a such that when the first partial sleeve member 89a is disposed about and/or around the first intermediate portion 87a of the first axial locking member 84a, and/or when the first intermediate portion 87a of the first axial locking member 84a is disposed within the concave portion of the first partial sleeve member 89a, the first axial locking member 84a (and the positioning tube 59 attached thereto) is axially secured to the handle assembly 40 and/or the tubular core member 39 (e.g., via the set screw 49, for example). Additionally, when the first partial sleeve member 89a is disposed about and/or around the first intermediate portion 87a of the first axial locking member 84a, and/or when the first intermediate portion 87a of the first axial locking member 84a is disposed within the concave portion of the first partial sleeve member 89a, the first axial locking member 84a (and the positioning tube 59 attached thereto) is rotationally decoupled from the handle assembly 40 and/or the tubular core member 39.

Similar to above, in some embodiments, the second axial locking assembly 83b may comprise a second partial sleeve member 89b configured to be positioned between the second proximal flange 85b and the second distal flange 86b of the second axial locking member 84b such that the second axial locking member 84b is rotatable relative to the second partial sleeve member 89b.

In some embodiments, the second partial sleeve member 89b may be generally concave in shape and/or may define a partial tube or a partial tubular structure about a longitudinal axis of the second partial sleeve member 89b. The second partial sleeve member 89b extends around and/or along less than 100% of a circumference around the longitudinal axis of the second partial sleeve member 89b. In some embodiments, the second partial sleeve member 89b may extend around and/or along at least 25% of a circumference around the longitudinal axis of the second partial sleeve member 89b. In some embodiments, the second partial sleeve member 89b may extend around and/or along at least 40% of a circumference around the longitudinal axis of the second partial sleeve member 89b. In some embodiments, the second partial sleeve member 89b may extend around and/or along at least 50% of a circumference around the longitudinal axis of the second partial sleeve member 89b. In some embodiments, the second partial sleeve member 89b may extend around and/or along at least 60% of a circumference around the longitudinal axis of the second partial sleeve member 89b. In some embodiments, the second partial sleeve member 89b may extend around and/or along at least 75% of a circumference around the longitudinal axis of the second partial sleeve member 89b.

In at least some embodiments, the second intermediate portion 87b of the second axial locking member 84b may be configured to extend within and/or pass through a concave portion of the second partial sleeve member 89b. In at least some embodiments, an outer diameter of the second proximal flange 85b may be greater than an inner diameter of the concave portion of the second partial sleeve member 89b, and an outer diameter of the second distal flange 86b may be greater than the inner diameter of the concave portion of the second partial sleeve member 89b. In some embodiments, the second axial locking assembly 83b may be disposed within the tubular core member 39 and/or the handle assembly 40. As discussed herein, the second extension 47 may be coupled with and/or secured to the inner shaft 54. In some embodiments, the second extension 47 may be coupled with and/or secured to the second axial locking assembly 83b. In some embodiments, the second extension 47 may extend radially outward from the second axial locking assembly 83b to engage with the second helical slot of the second translation mechanism 46. In some embodiments, an outer surface of the second partial sleeve member 89b may comprise an angled groove formed therein. In some embodiments, the second partial sleeve member 89b may comprise a threaded aperture formed therein. In some embodiments, the second extension 47 may be configured to engage with the threaded aperture formed in the second partial sleeve member 89b.

In some embodiments, the second partial sleeve member 89b may be axially trapped between the second proximal flange 85b and the second distal flange 86b such that when the second partial sleeve member 89b is disposed about and/or around the second intermediate portion 87b of the second axial locking member 84b, and/or when the second intermediate portion 87b of the second axial locking member 84b is disposed within the concave portion of the second partial sleeve member 89b, the second axial locking member 84b (and the inner shaft 54 attached thereto) is rotationally decoupled from the handle assembly 40 and/or the tubular core member 39.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle assembly, the elongate shaft assembly, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An implant delivery system for delivering a replacement heart valve implant to a native heart valve, comprising:
a handle assembly; and
an elongate shaft assembly extending distally from the handle assembly;
wherein the elongate shaft assembly comprises:
an inner shaft axially secured to the handle assembly;
an outer tubular member axially secured to the handle assembly and disposed about the inner shaft; and
an implant holding portion configured to constrain the replacement heart valve implant in a radially collapsed configuration when the implant holding portion is disposed in a delivery configuration, wherein the implant holding portion is rotatable relative to the outer tubular member.

2. The implant delivery system of claim 1, wherein the outer tubular member is rotatable relative to the handle assembly.

3. The implant delivery system of claim 1, further comprising at least one axial locking assembly configured to rotationally decouple the inner shaft and the outer tubular member from the handle assembly.

4. The implant delivery system of claim 1, further comprising an intermediate tubular member fixedly secured to the handle assembly;
wherein the inner shaft is movably disposed within the intermediate tubular member and the outer tubular member is movably disposed about the intermediate tubular member.

5. The implant delivery system of claim 1, wherein the outer tubular member is rotatable relative to the inner shaft.

6. The implant delivery system of claim 1, wherein the handle assembly is configured to axially move the inner shaft relative to the outer tubular member.

7. The implant delivery system of claim 1, wherein the implant holding portion comprises a proximal sheath axially secured to the outer tubular member and a distal sheath axially secured to the inner shaft.

8. The implant delivery system of claim 7, wherein the proximal sheath is axially movable relative to the distal sheath to shift the implant holding portion from the delivery configuration to a release configuration.

9. The implant delivery system of claim 1, further comprising a positioning tube disposed about the outer tubular member, wherein the positioning tube is axially secured to the handle assembly.

10. A replacement heart valve system, comprising:
a replacement heart valve implant comprising an expandable framework configured to shift from a radially collapsed configuration to a radially expanded configuration, and a plurality of valve leaflets disposed within and secured to the expandable framework; and
an implant delivery system comprising a handle assembly and an elongate shaft assembly extending distally from the handle assembly;
wherein the elongate shaft assembly comprises:
an inner shaft axially secured to the handle assembly;
an outer tubular member axially secured to the handle assembly and disposed about the inner shaft; and
an implant holding portion;
wherein the implant holding portion is configured to constrain the replacement heart valve implant in the radially collapsed configuration when the implant holding portion is disposed in a delivery configuration, wherein the implant holding portion is rotatable relative to the outer tubular member.

11. The replacement heart valve system of claim 10, wherein the replacement heart valve implant is configured to shift toward the radially expanded configuration when the implant holding portion moves from the delivery configuration to a release configuration.

12. The replacement heart valve system of claim 10, wherein the elongate shaft assembly comprises a distal tip fixedly secured to a distal end of the inner shaft.

13. The replacement heart valve system of claim 12, wherein the implant holding portion comprises a proximal sheath axially secured to the outer tubular member and a distal sheath axially secured to the distal tip;
wherein in the release configuration a distal end of the proximal sheath is spaced apart from a proximal end of the distal sheath by a greater distance than in the delivery configuration.

14. The replacement heart valve system of claim 10, wherein the elongate shaft assembly further comprises a positioning tube disposed about the outer tubular member, wherein the positioning tube is axially secured to the handle assembly.

15. A method of delivering a replacement heart valve implant to a native heart valve, comprising:
advancing an implant delivery system in a delivery configuration to a position adjacent the native heart valve, wherein the implant delivery system comprises a handle assembly and an elongate shaft assembly extending distally from the handle assembly, the elongate shaft assembly comprising an inner shaft axially secured to the handle assembly, an intermediate tubular member fixedly attached to the handle assembly, and an outer tubular member axially secured to the handle assembly;
wherein the replacement heart valve implant is constrained within an implant holding portion of the implant delivery system in a radially collapsed configuration when the implant delivery system is disposed in the delivery configuration;
rotating the handle assembly of the implant delivery system to rotate the replacement heart valve implant within the native heart valve without rotating the outer tubular member; and
deploying the replacement heart valve implant within the native heart valve.

16. The method of claim 15, wherein rotating the handle assembly of the implant delivery system causes the intermediate tubular member to rotate relative to the outer tubular member.

17. The method of claim 15, wherein the implant holding portion comprises a proximal sheath and a distal sheath configured to move axially away from each other to deploy the replacement heart valve implant.

18. The method of claim 15, wherein rotating the handle assembly of the implant delivery system causes the intermediate tubular member to rotate relative to the inner shaft.

19. The method of claim 15, wherein the distal sheath is axially secured to the inner shaft and the distal sheath is configured to rotate relative to the inner shaft.

20. The method of claim 15, wherein the proximal sheath is axially secured to the outer tubular member and the proximal sheath is configured to rotate relative to the outer tubular member.
